(19) United States
(12) Patent Application Publication
(10) Pub. No.: US 2019/0282590 A1
(43) Pub. Date: Sep. 19, 2019

(54) MEDICAMENT FOR TREATMENT AND/OR PROPHYLACTIC TREATMENT OF BREAST CANCER

(71) Applicant: HAKMED AB, Lund (SE)

(72) Inventor: Håkan OLSSON, Lund (SE)

(21) Appl. No.: 16/300,653

(22) PCT Filed: May 12, 2017
(86) PCT No.: PCT/EP2017/061481

§ 371 Date: Nov. 12, 2018

(30) Foreign Application Priority Data
May 13, 2016 (SE) 1650646-1

(51) Int. Cl. A61K 31/58; A61P 35/00

(52) U.S. Cl. CPC A61K 31/58; A61P 35/00

(57) ABSTRACT

A composition for use in the treatment and/or prophylactic treatment of breast cancer includes a 5-Alpha Reductase Inhibitor (5-ARI) and an excipient.

### FIELD OF THE INVENTION

This invention pertains in general to the field of male breast cancer treatment. More particularly the invention relates to a medicament for treatment and prophylactic treatment of breast cancer.

### BACKGROUND OF THE INVENTION

It is known that breast cancer is a rare malignancy in men, evidenced by the fact that there were only 45 reported new cases in Sweden during 2011, according to a cancer epidemiology report from The National Board of Health and Welfare (Socialstyrelsen). In fact, male breast cancer (MBC) accounts for only around 0.1% of the new cases of cancer among men and just above 0.5% of all yearly breast cancer cases in Sweden. Previous reports however suggest that MBC accounts for 1% of the breast cancer cases in the United States and that the incidence of MBC is increasing. MBC is a severe disease with high mortality and morbidity and the high mortality is believed to be caused by the older age of the patients at the time of diagnosis. Though the rarity of MBC still seems undeniable, the combination of the reported increase in incidence and the high mortality rate is alarming.

Due to its rarity, the etiology of MBC is still largely unidentified and poorly characterized, though genetic, environmental and hormonal factors are believed to play a role in the pathogenesis of the disease. As such, there is a need for new treatments and preventive measures for male breast cancer.

### SUMMARY OF THE INVENTION

It is an object of the present invention, considering the disadvantages mentioned above, to provide a pharmaceutical composition, comprising a 5-Alpha Reductase Inhibitor (5-ARI) and an excipient, for use in the treatment and/or prophylactic treatment of breast cancer.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a bar chart displaying the distribution of MBC by year of diagnosis,

FIG. 2 is a box plot of the ages of the cases at the time of diagnosis, and

FIG. 3 is a curve presenting the Kaplan-Meier estimate of the survival analysis of the included subjects after implementation of the exclusion criteria, analyzing the proportion at risk correlated to the time after 1^{st} of July 2005.

### DESCRIPTION OF EMBODIMENTS

The following description focuses on an embodiment of the present invention applicable to a pharmaceutical composition for use in the treatment and prophylactic treatment of male breast cancer. However, it will be appreciated that the invention is not limited to this application but may be applied to many other breast cancer types as well, including for example androgen receptor-positive (AR+) breast cancer.

Due to its rarity, the etiology of MBC is still largely unidentified and poorly characterized. Genetic, environmental and hormonal factors are believed to play a role in the pathogenesis of the disease.

The most common type of MBC is the invasive ductal carcinomas, which accounts for about 90% of all male breast tumors making MBC. While intraductal cancer, inflammatory carcinoma, and Paget's disease of the nipple have been described, lobular carcinoma in situ has not been seen in males. Although MBC in many cases seem histologically similar to female breast cancer (FBC), MBC and FBC differ in the sense that MBC tumors express estrogen (ER) and progesterone (PR) receptors more frequently than FBC tumors and HER2 is less frequently over-expressed or amplified. Also, only about 1% of the MBCs are triple-negative (estrogen-receptor-negative, progesterone-receptor-negative, and HER2-negative), while in women 10%-15% of FBCs are triple-negative. According to existing publications, 80-90% of male breast cancers express estrogen receptors and 74-81% express progesterone receptors. Thus, it can be concluded that MBC and FBC differ on a molecular level and that the androgen receptor (AR) appears to have a more central role in the etiology of MBC, with around 87% of the MBC tumors expressing AR. Taken together, the high rates of expression of hormone receptors makes the hormone-affecting approach on the MBC interesting, not least from statistical and epidemiological perspectives. This also explains why it is common that results from breast cancer studies, such as using mouse and cellular lobular breast cancer (the most common special breast cancer (BC) subtype) models, cannot be applied to male breast cancer. This also explains why MBC treatments cannot be successfully modeled on FBC.

Studies on morphological changes in the female breast have shown that the risk of developing breast cancer is likely correlated to the amount of breast tissue available for undergoing carcinogenic transformation. This knowledge might be applicable to studies of MBC, since occurrence of gynecomastia may increase the amount of breast tissue available for carcinogenic transformation and thus indirectly predispose for MBC. Gynecomastia is characterized as a benign uni- or bilateral condition with increased size of the male breast tissue due to hyperplasia of the ductal and stromal breast tissue and is linked with estrogen excess. Therefore, it is necessary to evaluate the risk of developing MBC after using medicines known to cause gynecomastia or hyperestrogenemia. However, there are several etiological causes of gynecomastia other than hormone-affecting exogenous drug use. Predisposing factors to gynecomastia that need to be taken into consideration are endogenous hormonal imbalance affecting the estrogen/androgen ratio seen in early puberty and late adulthood as well as damage to the liver or gonads also affecting this hormonal ratio. Since gynecomastia commonly is a benign and often times temporary condition, it is debatable whether gynecomastia should be considered primarily as a predisposing factor to MBC, a symptom of MBC or a benign condition, when it can appear to be all of these three alternatives. The existing literature is still not conclusive in this matter.

Several medicines are known to cause gynecomastia by already plotted hormone-affecting mechanisms; among these are estrogen-containing and estrogen-like medications, medicines stimulating estrogen synthesis (e.g. gonadotropins and growth hormone), aromatizable estrogen precursors (e.g. exogenous androgens and androgen precursors), medicines causing direct testicular damage (e.g. busulfan, nitrosurea, vincristine and ethanol), medicines blocking testosterone synthesis (e.g. ketoconazole, spironolactone, metronidazole and etomidate) or androgen action (e.g. flutamide, bicalutamide, finasteride and cyproterone), as well as medicines displacing estrogen from SHBG (e.g. spironolactone and ethanol).

This knowledge shows that medications affecting the hormonal balance by raising estrogen levels or decreasing testosterone levels, alternatively blocking the effect of testosterone, may cause growth of the breast tissue and through this has been proposed to possibly enhance the risk of carcinogenic transformation. The presumption is that the aforementioned medicines may increase the risk of developing MBC through increasing the amount of breast tissue with high estrogen/androgen ratio being the common denominator. Some of these medicines have already been proposed to increase the risk of MBC, such as anti-androgens probably primarily through altered estrogen/androgen ratio and induced hyperestrogenic environment. This, combined with the suggested 87% AR expression among MBC tumours, proposes a possibly strong correlation between androgens and MBC. High frequency expression of AR in MBC cells offers suspicion of the possibly central role of androgens in the MBC pathogenesis and has historically driven the hypothesis that suppression of androgen production or blockage of androgen receptors might be a successful way to treat MBC.

Triple-negative breast cancer (sometimes abbreviated TNBC) refers to any breast cancer that does not express the genes for estrogen receptor (ER), progesterone receptor (PR) or Her2/neu, making it difficult to treat with conventional chemotherapies targeting one of the three receptors. A subset of TNBC patients (0-53% of all TNBC patients depending on study population) has been reported to express androgen receptor (AR) in carcinoma cells and the manipulation of androgen signalling or AR targeted therapies have been proposed. The high AR expression among MBC tumours and possibly central role of androgens in the MBC pathogenesis suggests that MBR therapies may also be applicable on androgen receptor-positive (AR+) breast cancers. This is especially interesting for the cancer forms difficult to treat with conventional therapies.

Of course, treating MBC with anti-androgens when it is believed to be caused by high estrogen/androgen ratio serves as somewhat of a paradox. This can be explained by clarifying that circulating androgens can affect MBC in two ways. Firstly, they do this by directly exerting tumor-promoting signaling on the AR-expressing MBC cells and thereby activating the intracellular pathways of breast cells with no prior exposure to anti-hormone treatment, thus acting with causing properties. Secondly, increased androgen levels during treatment with aromatase inhibitors (AI) represent excess substrate for aromatization and outcompetes the effects of AIs, which is why androgen inhibition results in tumor shrinkage and can be therapeutic in MBC cases

Sporadic associations with MBC have been reported regarding 5-α reductase inhibitors (5-ARI) (Shen C. Lee et. al. J. Nat. Cancer Inst., Vol. 96, No. 4, Feb. 18, 2004), since 5-ARI medication obstructs the conversion of testosterone to the more potent dihydrotestosterone (DHT) through inhibition of steroid 5-α reductase and thereby causes an increased estrogen/androgen ratio. However, recent studies have observed no increased risk of MBC in men using 5-α reductase inhibitors.

Epidemiological studies of MBC are uncommon, with mostly small case-control studies published, and even fewer studies have been made exploring the possibility of exogenous substance use causing MBC. Compared to FBC, MBC is a relatively unexplored fraction of breast cancer, most likely due to limitations regarding the sizes of data sets and far greater amounts of female cases and therefore greater material of FBC in comparison. The difference in research quantity between MBC and FBC can easily be visualized by a couple of PubMed searches, showing 1079 hits on the search term “male breast cancer”, whilst searching for “breast cancer” alone generated 195 969 hits on 2015 Nov. 2. However, if these numbers are correlated to the amount of cases of breast cancer for each sex, the distribution of publications is quite reasonable. The difference is visualized through published reports that present an incidence of MBC generally less than 1 per 100,000 men per year and with a female-to-male incidence rate ratio of 122, representing around 1% of all malignant breast tumors. To this date, FBC research has overwhelmed MBC and often times served as a guide for clinical treatment of MBC, even though there are several differences between FBC and MBC.

Database mining has proven a very powerful tool for finding unseen trends in treatment results. The Swedish Drug Register contains data on pharmaceuticals, supplies, and food taken prescription or equivalent in pharmacy from 1999 onwards. The number of prescriptions is almost 100 million a year. The register is updated with new information every month. The Swedish Cancer Registry was founded in 1958 and covers the entire population of Sweden. Approximately 50 000 malignant cases of cancer is registered every year in Sweden. It is compulsory for every health care provider to report newly detected cancer cases to the registry. A report has to be sent for every cancer case diagnosed at clinical-, morphological-, other laboratory examinations as well as cases diagnosed at autopsy. Thus, these registers provide a very good foundation for database mining seen from an international standard.

In this retrospective case-control cancer epidemiological register study we aim to turn the tables and use MBC data to further examine the role of medication in breast cancer etiology in general. We accepted the rough fragmentation regarding gender with cases and controls recorded as “male” being included, not considering the possibility of that included cases or controls might have undergone sex change. Though the term “male breast cancer” is frequently used in this study and only individuals recorded as “male” were included to shed light on the etiology of this severe disease. The aim with this register study is to map possibly enhanced statistical risk of incurring MBC in men using drugs affecting the male hormonal state and causing gynecomastia, and by this possibly obtain better knowledge regarding the etiology of MBC for future studies. The results of this study may be applicable to breast cancer research regardless of gender, if the results point at a certain medicine or category of medicines with causing or protective properties correlated to the risk of contracting MBC and thereby secondarily bring hints regarding the etiology of breast cancer.

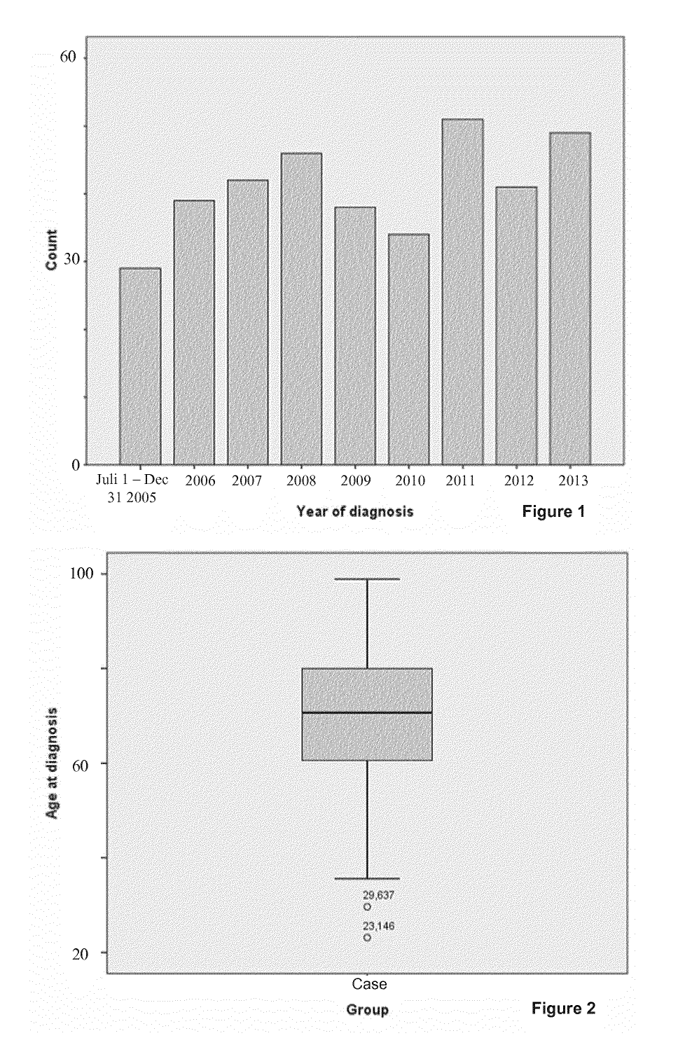

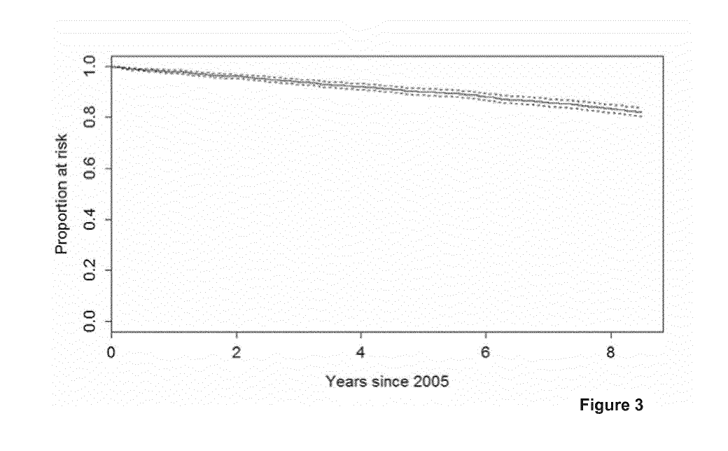

Even so, it is extremely hard to get reliable results for a condition with such small number of annual cases. Thus, it has taken a population-wide study to show concrete results for the role of medication in male breast cancer. As can be seen in FIGS. 1 and 2, even population-wide data over a period of over 8 years only generated descriptive statistics for 369 cases, including the ones diagnosed with prostate cancer. FIGS. 1 and 2 visualize the distribution of cases by year of diagnosis and the ages of the cases at the time of diagnosis. A survival analysis of the included subjects after implementation of the exclusion criteria (explained under material and methods below) was also performed, analyzing the proportion at risk correlated to the time after 1st of Jul. 2005. The Kaplan-Meier estimate was used and the curve is presented in FIG. 3.

Thus, when conduction a national population based study relating to male breast cancer patients, the present inventors found the surprising result that treatment using 5-ARI (5-Alpha Reductase Inhibitor) has a positive effect on treatment and prophylactic treatment of male breast cancer. After exclusion of the prostate cancer cases, 2992 subjects remained, 330 of these were MBR cases. First, we analyzed the earliest prescribed medicine of each included subject. In order to specify the usage of medications and to possibly observe patterns regarding the risk of MBC development, the medications were grouped in seven groups. 116 of the subjects were had 5-ARIs as the first prescribed medicine and this group of men displayed non-significant HR 0.39 (95% CI 0.14-1.10) using conditional logistic regression. The Cox regression gave similar results: HR 0.38 (95% CI 0.14-1.02), still non-significant with p=0.055). Thus, the national population based study of male breast cancer and matched controls deriving drug exposure from the Swedish Drug Prescription Registry found that in men with Benign prostatic hyperplasia (BPH), the risk of male breast cancer was significantly reduced in 5-ARI users compared with other men with BPH, who have been found to in our and in other studies to have an increased risk of male breast cancer. As a comparative example, the study also showed that the risk of MBC was statistically significantly higher among men using αARAs, as has previously been shown for BPH. Alfuzosin and Terazosin, both αARAs, showed significant correlation to increased risk of MBC. These medications are used to treat moderate to severe symptoms of BPH by selective blockage of postsynaptic al-receptors. In this study we found that the risk of contracting MBC was statistically significantly associated with use of αARAs.

Thus one embodiment relates to a pharmaceutical composition, comprising a 5-Alpha Reductase Inhibitor (5-ARI) and an excipient, for use in the treatment and/or prophylactic treatment of breast cancer. 5-ARI's are a class of drugs with antiandrogen effects, and refer to drugs which inhibit one or more isoforms of the enzyme 5α-reductase.

5α-reductase inhibitors, such as finasteride, dutasteride, and alfatradiol, are antiandrogenic as they prevent the conversion of testosterone, the major androgen sex hormone, to dihydrotestosterone (DHT). DHT is 3-5 times more potent than testosterone or other androgens (except in skeletal muscle tissue, where testosterone is the main androgen). The systematic (IUPAC) name för dutasteride is (5α,17β)-N-{2,5-Bis(trifluoromethyl)phenyl}-3-oxo-4-azaandrost-1-ene-17-carboxamide and N-(1,1-dimethylethyl)-3-oxo-(5α,17β)-4-azaandrost-1-ene-17-carboxamide for finasteride.

In one embodiment, the 5-ARI of said pharmaceutical composition is dutasteride, finasteride, alfatradiol, or a combination thereof. The study seems to point to that the preventive effect was strongest for dutasteride users. Thus, in one embodiment, the 5-ARI is dutasteride.

5α-reductase inhibitors are unique because they do not counteract the effects or production of other androgens other than DHT. Dihydrotestosterone is necessary for development of both external male sex organs and the prostate. 5α-reductase inhibitors are most often used to treat benign prostatic hyperplasia since the resulting decrease in dihydrotestosterone inhibits proliferation of prostate cells. Beyond being a catalyst in the rate-limiting step in testosterone reduction, 5α-reductase isoforms I and II reduce progesterone to 5α-dihydroprogesterone (5α-DHP) and deoxycorticosterone to dihydrodeoxycorticosterone (DHDOC). 5α-DHP is an agonist of the progesterone receptor.

In one embodiment, the breast cancer is male breast cancer and/or androgen receptor-positive (AR+) breast cancer. In one further embodiment, the male breast cancer is selected from the group consisting of invasive ductal carcinoma (IDC), invasive medullary, invasive mucinous, ductal cancer in situ (DCIS), micropapillary, papillary, and tubular cancer.

Single daily doses of dutasteride up to 40 mg/day (80 times the therapeutic dose) have been administered for 7 days without significant safety concerns to healthy volunteers. In clinical studies, doses of 5 mg daily have been administered to patients for 6 months with no additional adverse effects to those seen at therapeutic doses of 0.5 mg.

The defined daily dose (DDD) is the assumed average maintenance dose per day for a drug used for its main indication in adults, as defined by the World Health Organization (WHO). The DDD provide a fixed unit of measurement independent of price and dosage form (i.e. tablet strength) and also reflects the potency of a drug. It is used to standardize the comparison of drug usage between different drugs or between different health care environments. It is a statistical measure of drug consumption and should not be confused with the dose actually prescribed by a physician for an individual patient.

Thus, in one embodiment, the daily dose of the dutasteride corresponds to the defined daily dose (DDD). In one embodiment, the daily dose of dutasteride is from 0.1 mg to 40 mg, preferably as 5 to 0.25 mg, or more preferably about 0.5 mg.

Single daily doses of finasteride up to 400 mg and multiple doses up to 80 mg have been administered daily for three months without subjects experiencing any side effects.

Thus, in one embodiment, the daily dose of the finasteride corresponds to the defined daily dose (DDD). In one embodiment, the daily dose of finasteride is from 1 mg to 400 mg, preferably from 2.5 mg to 80 mg, or more preferably about 5 mg.

An excipient is a natural or synthetic substance formulated alongside the active ingredient of a medication. Purpose includes stabilization, bulking, facilitating drug absorption, reducing viscosity, or enhancing solubility. Excipients may also be useful for the manufacturing process, to aid in the handling of the active substance. The selection of appropriate excipients also depends upon the route of administration and the dosage form, as well as the active ingredient and other factors. In one aspect of the invention, the pharmaceutical composition comprises an excipient selected from the list containing lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, polyethylene glycol, poloxamers, lactose, hydroxypropylmethylcellulose (HPMC), hydroxypropyl cellulose (HPC), lactose, and polyvinylpyrrolidone. In one further aspect of the invention, the 5-ARI is administered orally.

Male carriers of a BRCA1, BRCA2, PALB2 mutation, especially BRCA2 mutation, experience an increased risk especially for male breast cancer and prostate cancer. Estimates have suggested a life time risk of 6-9% for male breast cancer and around 30% for prostate cancer. Also a worse prognosis of prostate cancer among carriers has been shown. Another genetic risk factor is the chromosomal deviation Klinefelter's syndrome.

In one embodiment, the prophylactic treatment with said pharmaceutical composition is for an individual being a carrier of a BRCA1, BRCA2, PALB2 mutation, or chromosomal deviation Klinefelter's syndrome or combinations thereof.

Other known risk factors for developing MBC are obesity, diabetes, physical inactivity, heavy alcohol consumption, exposure to ionizing radiation and late onset of puberty. Conditions affecting the hormonal levels directly or indirectly are also linked with MBC, for example liver disease, like liver cirrhosis, previous testicular pathology and high levels of estradiol and plasma prolactin. A hormonally oriented etiology of breast cancer among men has also been described, more specifically an imbalance in the estrogen/androgen ratio as the common denominator through androgen deficiency or via relatively high levels of endogenous estrogens leading to high estrogen/androgen which secondarily may cause gynecomastia of the patients breast tissue. Gynecomastia is also marked as a risk factor for contracting MBC, primarily as a consequence of altered hormonal ratio and high estrogen levels. Usage of exogenous androgens and estrogens, and possibly other medications that alter men's hormonal levels are also considered possible risk factors.

In one embodiment, the prophylactic treatment with said pharmaceutical composition is preferably for an individual with known risk factors for developing MBC selected from the group consisting of obesity, diabetes, physical inactivity (less than WHO recommendations), heavy alcohol consumption (such as more than about 150 g ethanol per week), exposure to ionizing radiation, late onset of puberty (no testicular enlargement by 14 years (male) or breast enlargement at 13 years (female)), liver disease (liver cirrhosis), testicular pathology, high levels of estradiol (>200 pmol/L) and plasma prolactin (>9 μg/L), imbalance in the estrogen/androgen ratio, gynecomastia of the breast tissue, and usage of exogenous androgens and estrogens.

5-ARI:s finasteride, have been shown to prevent prostate cancer. Also for dutasteride a better prognosis has been shown. It has been found that the prevention has mainly been limited to less severe prostate cancer raising the possibility of a detection bias or that the agents worsen the prognosis for some cases. However, it suggests that prophylactic or preventive treatment of male breast cancer using said medical composition will be especially beneficial for risk groups with higher risk of both MBR and prostate cancer.

Thus in one embodiment, a medical composition comprising 5-ARI (5-Alpha Reductase Inhibitor) is for use in the treatment and prophylactic treatment of both male breast cancer and prostate cancer.

An alternative embodiment relates to the use of a 5-Alpha Reductase Inhibitor (5-ARI) and an excipient, for the manufacture of medicament for use in preventing, or reducing the risk of developing, breast cancer in a human being. The medicament is to be orally administrated to the human being. Yet another alternative embodiment relates to a method for preventing, or reducing the risk of developing, breast cancer in a human being, the method comprising mucosally administering the 5-Alpha Reductase Inhibitor (5-ARI) and an excipient to a human being. Aspects provided below in relation to a 5-Alpha Reductase Inhibitor (5-ARI) selected from the group consisting of dutasteride, finasteride, alfatradiol, or a combination thereof, and an excipient for use in preventing, or reducing the risk of developing, breast cancer in a human being, are equally applicable to these alternative embodiments.

An alternative embodiment relates to a method for treatment and/or prophylactic treatment of breast cancer, comprising the steps of, identifying an individual with a genetical predisposition for male breast cancer, and administering to the individual a pharmaceutical composition comprising a pharmaceutical active amount of 5-Alpha Reductase Inhibitor (5-ARI) and an excipient.

Material and Methods

Since July 2005 369 cases of MBC were identified from the Swedish Cancer Register and each case was matched to five controls (n=2971) from the general population of the same age and domicile. Medicines with documented side effects of hormonal imbalance or gynecomastia were collected from the Prescribed Drug Register in operation since July 2005. Drug exposure was followed both for cases and controls until the cancer diagnosis or death of the case. Cases and controls with a diagnosis of prostate cancer were excluded. The data were mainly analyzed with conditional logistic regression and Cox regression.

Study Population

The cases were received from the Swedish Cancer Register (Cancerregistret). The total number of cases with a breast cancer diagnosis in the register, as recorded by the International Classification of Diseases, 7th Edition (ICD-7), was 104 045. These cases included both men and women, in the years of 2000-2013.

The identified case group was sent to the Swedish agency Statistics Sweden (Statistiska Centralbyrån, SCB) to randomly be matched to five controls from the General Population Register according to birth year, sex and county, with an additional condition in the matching process being that the controls had to be alive at the date of cancer diagnosis of the matched case. Therefore, the material included a matched control group created by SCB, with each index case plus the five matched controls constituting a match set. In total the case file included 104 045 unique persons with 104 278 entries. Each case and control received an individual identification (ID) number and this ID number was the same in all databases. This was a crucial tool in the matching process and the forming of the cohort.

The key from SCB with the controls included contained 628 061 unique persons with 624 214 ID numbers, still including both men and women. These 628 061 persons were matched with the Patient Register (Patientregistret, 1964-2013), the Prescribed Drug Register (Läkemedelsregistret, 2005-2015), the Cancer Register (Cancerregistret, 1958-2013) and the Death Register (Dödsorsaksregistret, 2000-) using the ID number and by this creating an anonymized cohort with a code from The National Board of Health and Welfare.

Through the ID number, it was also possible to match the data to the Non-Institutional Care Register (Öppenvårdsregistret, 1995-), the Residential Care Register (Slutenvårdsregistret, 1995-) and the Surgical Register (Operationsregistret, 2000-). The material was sent to the Department of Cancer Epidemiology in Lund and included the registered cases of MBC in Sweden between the years of 2000 and 2013 used in this study.

For a case to be included in this study it had to be a male diagnosed with MBC (International Classification of Diseases, 7th edition: diagnose codes 1701-02 and 1707-09) sometime between 2000 and 2013, regardless of whether or not MBC was diagnosed as the first cancer in order to maximize the number of cases. A total of 595 cases of MBC (n=595) and 2971 matched controls were received, thus a mean of 42.5 cases of MBC per year over the fourteen years of material.

Identifying Medical Exposure

Initially medications with gynecomastia as a known side effect were identified through literature searches in PubMed with a subsequent literature review of publications regarding the etiology of MBC, and lastly through the Swedish overview of registered medications, Farmaceutiska Specialiteter i Sverige (FASS). The Anatomic Therapeutic Chemical (ATC) classification system was used to detect the relevant medicines in the Prescribed Drug Register. The medicines known to cause gynecomastia that were selected and included in the search of prescriptions in the Prescribed Drug Register are displayed in Table 1.1.

By extracting prescriptions of these medications from the Prescribed Drug Register using the ATC codes and the ID numbers of each included case and control, a database with usage of the listed medications was generated. This database identified each individual through the ID number and each separately collected prescription was listed as a single case, thus identifying usage of these medications among cases and controls. This extraction identified 78 287 collected prescriptions among the cases and controls from 1st of Jul. 2005 (when the Prescribed Drug Register was introduced) to 31st of Dec. 2013, dispersed over 2404 individuals, leaving 1162 of the men in this study without exposure to any of these medicines in this period of time.

Initial Processing of the Data

The data from the extraction of the Prescribed Drug Register were matched to the Cancer and Patient Registers, creating a dataset with information on whether each individual was a case or control, as well as their status regarding ever-use of the included medications listed in Table 1.1. This was performed by merging the file containing the extraction from the Prescribed Drug Register with the Cancer and Patient Registers through matching each individual's unique ID number. This resulted in a new dataset containing 595 cases and 2671 controls, with variables showing case or control status, ever-use of at least one of the included medications after the 1st of Jul. 2005 as well as all specific medicines prescribed to each individual stated and listed as ATC codes.

The variable containing ATC codes of the specific prescriptions was used to create one variable for each specific medicine, giving patients that never used the specific medicine the value 0 and patients with recorded use of the specific medicine the value 1. Consequently, men with no use of these medications got zeros in each variable obtained from the Prescribed Drug Register. Through this, it was possible to distinguish the usage of the included medications among cases and controls respectively, though the match sets created by Statistics Sweden at this point were dissolved. Also, in this first test regarding ever-use of the selected medicines, no exclusion of medication prescribed after the case's date of diagnosis was implemented and cases diagnosed before 1st of Jul. 2005 were not excluded.

These preparations and statistical tests were merely a way to get an initial overview of the included medications and served as a guide for later refined statistical testing. In this way, by creating cross tables comparing cases with controls, it was possible to get an overview of whether or not the ever-use of included medications correlated to the case or control status. The processing of the data was performed in the IBM SPSS Statistics Data Editor, 22nd Edition. The statistical results of the cross tabulations were displayed in Chi square including Fisher's Exact Test and were performed using SPSS.

Statistical Analysis and Study Design

Cox regression and nested case-control analysis were used in analysis of the ever-use variable of any of the included medicines, after the exclusion phases (described below), as well as after the grouping phase and generated study design-specific hazard ratios (HR) and 95% confidence intervals (CI). P-values less than 0.05 were considered statistically significant. All processing of the data and statistical analysis was made using R programming for statistical computing and ended with the effectuation of conditional logistic regression, Cox regression and nested case-control. Using multiple statistical methods provided additional statistical certainty.

Exclusion

To refine the dataset for further statistical analysis, all cases diagnosed prior to the introduction of the Prescribed Drug Register in 2005 were excluded, since the data entries regarding usage of medications only stretch back to the 1st of Jul. 2005. The complete exposure of all individuals prior to this date was impossible to obtain. The date of entry was set to 1st of Jul. 2015 if the subject had medications prescribed this day and therefore got classified as exposed. Date of entry could of course be a later date depending on when the patient collected one of the medicines included in the study.

In this second line of statistical analysis, the match sets created by Statistics Sweden were kept, using the ID number of the case to connect the five matched controls to each case. All medical use after the time of diagnosis of the case was excluded, which applied also to the controls, meaning that the matched control's medical usage after the date of diagnosis of the case was censored using the predefined match sets. All subjects were classified as non-users until the date of the first prescription of one of the included medicines.

Data involving the date of death were received from the Death Register and used to define a date of exit regarding deceased individuals. Subjects who were alive on 31st of Dec. 2013, the end date of the follow-up in the Swedish Cancer Register, were automatically set for a date of exit on this date. The date of exit was therefore defined as; (1) the date of diagnosis of the case, (2) the date of death (which automatically ended the follow up) or (3) at the latest the 31st of Dec. 2013 (which was the overall end of follow-up), whichever occurred first. This exclusion was performed by determining the age and creating age variables of the cases at the time of diagnosis, as well as the age of each individual at the time of each prescribed medication.

After implementation of these exclusion criteria, 369 cases and 2971 controls remained and were included in the continuation of the study. Conditional logistic regression allowed for the use of the predefined match sets with controls five controls linked to each case. Cox regression dissolved the match sets created by Statistics Sweden, but enhanced the number of used observations in the statistical estimate to 5484. In the nested case-control analysis four controls were matched to each case, also dissolving the match sets created by Statistics Sweden and creating a cohort with a 4:1 control-to-case ratio. The control subjects were matched to the case patients based on age and medical exposure. 369 cases remained after exclusion, yielding 1496 controls using this method. The nested case-control design was only used in the ever-use analysis and was not applied after the grouping phase.

Grouping

In order to further specify the usage of medications and to possibly observe patterns regarding the risk of MBC development, the medications were grouped based on the results regarding frequencies of use among cases and controls and significant risk in the cross tabulations. The medications were grouped in seven groups, using the results from the Chi squares of the cross tabulations to create coherent groups with enough hits on usage to provide statistical analyzability. Subjects with no use of the included medications were assigned to group 0.

Group 0: non-users of the included medications in this study.

Group 1: C08CA Dihydropyridine derivatives.

Group 2: G04BE Medicines to treat erectile dysfunction.

Group 3: G04CB Testosterone 5α reductase inhibitors.

Group 4: H02AB Glucocorticoids.

Group 5: L02AA Estrogens, L02AE Gonadotropin releasing hormone agonists and L02BB Antiandrogens.

Group 6: N05AA Phenothiazins with aliphatic functional groups, N05AB Phenothiazins with piperazine rings, N05AD Butyrophenone derivatives, N05AE Indole derivatives, N05AF Thioxanthene derivatives, N05AH Dibenzodiazepines, dibenzoxazepines and dibenzothiazines, N05AN Lithium, N05AX Other antipsychotics and N05BA Benzodiazepine derivatives.

Group 7: including the remaining classes of the other included medicines with non-significant results in the cross tabulations or with few/none hits on usage.

The first prescribed medicine for each individual with recorded medical use was selected, since this was likely to be the medicine each individual had used for the longest period of time. After this grouping phase, conditional logistic regression, Cox regression and nested case-control were used to perform risk analyses of the dataset.

Exclusion of Prostate Cancer

In order to analyze the material more specifically regarding the effect of 5-α reductase inhibitors on the statistical risk of contracting MBC, the cases of prostate cancer (ICD-7 code 177) in this material were excluded due to 5-α reductase inhibitors being a frequently used medication among prostate cancer sufferers. The users of 5-α reductase inhibitors were grouped into the aforementioned group 3. With these individuals excluded 330 cases and 2662 controls remained in the dataset for analysis with conditional logistic regression and Cox regression.

Fragmentation of Group 7

After observing statistically significant results for group 7, this group was recoded and fragmented into four subgroups to possibly determine what might be the cause of these significant results, once again using the ATC codes for medical identification. Group 7 were subdivided into “Medicines to treat incontinence and for control of urination frequency” (ATC code G04BD), “α-adrenergic receptor antagonists” (ATC code G04CA), “Selective serotonin reuptake inhibitors” (ATC code N06AB) and one group including all other medications in group 7 (all other present ATC codes). A summary of the new groups, in this phase of the study, follows here.

Group A: including all use of medicines in group 1 to 6.

Group B: including all use of medicines other than these in group C-E.

Group C: including all use of medicines to treat incontinence and for control of urination frequency.

Group D: including all use of α-adrenergic receptor antagonists.

Group E: including all use of selective serotonin reuptake inhibitors.

Additional statistical analyses, including conditional logistic regression and Cox regression, were performed after this fragmentation using the whole dataset with 369 included MBC cases and 2971 controls, therefore not taking prostate cancer cases into consideration.

Statistical Analysis, Study Design and Programming Tool

It was determined that conditional logistic regression, Cox regression and nested case-control analysis all were applicable and useful statistical methods in this study. These statistical methods were used in for analyses after the exclusion phase and generated study design-specific hazard ratios (HR) and 95% confidence intervals (CI). P-values less than 0.05 were considered statistically significant.

Conditional logistic regression allowed for the use of the predefined match sets with five controls linked to each case. Cox regression dissolved the match sets created by Statistics Sweden, but enhanced the number of used observations in the statistical estimate to 5484. In the nested case-control analysis four controls were matched to each case, also dissolving the match sets created by Statistics Sweden and creating a cohort with a 4:1 control-to-case ratio. The control subjects were matched to the case patients based on age and medical exposure. 369 cases remained after exclusion, yielding 1496 controls using this method.

All processing of the data and statistical analysis was made using R programming for statistical computing and ended with the effectuation of conditional logistic regression, Cox regression and nested case-control. The conditional logistic regression was performed with function clogit from the package survival in R version 3.2.1. Using multiple statistical methods provided additional statistical certainty.

Results

Descriptive statistics of the 369 cases, including the ones diagnosed with prostate cancer, are presented in FIGS. 1 and 2. The purpose is to visualize the distribution of cases by year of diagnosis and the ages of the cases at the time of diagnosis. A survival analysis of the included subjects after implementation of the exclusion criteria was also performed, analyzing the proportion at risk correlated to the time after 1st of Jul. 2005. The Kaplan-Meier estimate was used and the curve is presented in FIG. 3.

Analysis of 5-ARI after Exclusion of Prostate Cancer Cases

After exclusion of the prostate cancer cases, 2992 subjects remained, 330 of these were cases. First, we analyzed the earliest prescribed medicine of each included subject. 116 of the subjects were had 5-ARIs as the first prescribed medicine and this group of men displayed non-significant HR 0.39 (95% CI 0.14-1.10) using conditional logistic regression. The Cox regression gave similar results: HR 0.38 (95% CI 0.14-1.02), still non-significant with p=0.05).

Analysis of αARA Before Exclusion of Prostate Cancer Cases

The initial analyses of αARA were performed prior to the exclusion of prostate cancer cases. The dataset contained a total of 3340 subjects, of which 369 were cases and 135 subjects had αARA as the earliest prescription. This group of subjects showed significant HR 2.20 (95% CI 1.35-3.59) when analyzed with the conditional logistic regression (p=0.002). Cox regression gave similar results: HR 2.24 (95% CI 1.48-3.39) and p<0.001.

Comparing 5-ARI with αARA users gave a reduced risk of (HR=0.17, CI 0.06-0.50). No other medication was significantly associated with an increased or decreased risk of MBC.

Although the present invention has been described above with reference to (a) specific embodiment(s), it is not intended to be limited to the specific form set forth herein. Rather, the invention is limited only by the accompanying claims and, other embodiments than the specific above are equally possible within the scope of these appended claims, e.g. different than those described above.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A method of treating or reducing the risk of breast cancer in a subject, the method comprising administering to the subject having breast cancer or at risk of breast cancer a 5-Alpha Reductase Inhibitor (5-ARI) and an excipient.

2. The method according to claim 1, wherein the 5-ARI is dutasteride, finasteride, alfatradiol, or a combination thereof.

3. The method according to claim 1, wherein the breast cancer is male breast cancer and/or androgen receptor-positive (AR+) breast cancer.

4. The method according to claim 2, wherein the male breast cancer is selected from the group consisting of invasive ductal carcinoma (IDC), invasive medullary, invasive mucinous, ductal cancer in situ (DCIS), micropapillary, papillary, and tubular cancer.

5. The method according to claim 1, wherein the 5-ARI is dutasteride and wherein the daily dose of dutasteride corresponds to the defined daily dose (DDD).

6. The method according to claim 5, wherein the daily dose of dutasteride is from 0.1 mg to 40 mg.

7. The method according to claim 1, wherein 5-ARI is finasteride, and wherein the daily dose of finasteride corresponds to the defined daily dose (DDD).

8. The method according to claim 7, wherein the daily dose of finasteride is from 1 mg to 400 mg.

9. The according to claim 1, wherein the treatment is prophylactic treatment, and wherein the subject to be prophylactically treated is a subject being a carrier of a BRCA1, BRCA2, PALB2 mutation, or chromosomal deviation Klinefelter's syndrome or combinations thereof.

10. The method of claim 1, wherein the treatment is prophylactic treatment, and wherein subject to be prophylactically treated is a subject with known risk factor(s) for developing male breast cancer, the risk factor(s) being selected from the group consisting of obesity, diabetes, physical inactivity, heavy alcohol consumption, exposure to ionizing radiation, late onset of puberty, liver disease (liver cirrhosis), testicular pathology, high levels of estradiol and plasma prolactin, imbalance in the estrogen/androgen ratio, gynecomastia of the breast tissue, usage of exogenous androgens and estrogens, and combinations thereof.

11. The method according to claim 5, wherein the daily dose of dutasteride is from 0.25 to 5 mg.

12. The method according to claim 5, wherein the daily dose of dutasteride is about 0.5 mg.

13. The method according to claim 7, wherein the daily dose of finasteride is from 2.5 mg to 80 mg.

14. The method according to claim 7, wherein the daily dose of finasteride is about 5 mg.
